SEQUENCE LISTING (50 sequences)

SEQ ID NO: 1 - DNA, 6 bp, Artificial Sequence
  Enhancer box (E-box)
cacgtg

SEQ ID NO: 2 - DNA, 8 bp, Artificial Sequence
  cAMP RE (CRE)
tgacgtca

SEQ ID NO: 3 - DNA, 9 bp, Artificial Sequence
  Amino acid RE (AARE)
attgcatca

SEQ ID NO: 4 - DNA, 8 bp, Artificial Sequence
  Forkhead box binding site (FBS)
ataaacaa

SEQ ID NO: 5 - DNA, 10 bp, Artificial Sequence
  Nuclear factor kappa B (NFkB-RE)
gggactttcc

SEQ ID NO: 6 - DNA, 15 bp, Artificial Sequence
  Nuclear factor 1 RE (NF1-RE)
ttggctatatgccaa

SEQ ID NO: 7 - DNA, 13 bp, Artificial Sequence
  Antioxidant RE (ARE)
atgacacagcaat

SEQ ID NO: 8 - DNA, 8 bp, Artificial Sequence
  Elongation factor 2 RE (E2F-RE)
tttcgcgc

SEQ ID NO: 9 - DNA, 9 bp, Artificial Sequence
  GC-box
ggggcgggg

SEQ ID NO: 10 - DNA, 9 bp, Artificial Sequence
  ETS binding site 1 (EBS1)
accggaagt

SEQ ID NO: 11 - DNA, 9 bp, Artificial Sequence
  ETS binding site 2 (EBS2)
acaggaagt

SEQ ID NO: 12 - DNA, 21 bp, Artificial Sequence
  Unfolded protein response element (UPRE)
gctgacgtggtgctgacgtgg

SEQ ID NO: 13 - DNA, 11 bp, Artificial Sequence
  Endoplasmic reticulum stress RE II (ERSE-II)
attggtccacg

SEQ ID NO: 14 - DNA, 9 bp, Artificial Sequence
  Yin Yang 1 RE (YY1-RE)
cgccatttt

SEQ ID NO: 15 - DNA, 19 bp, Artificial Sequence
  Endoplasmic reticulum stress RE (ERSE)
ccaatggccagcctccacg

SEQ ID NO: 16 - DNA, 10 bp, Artificial Sequence
  Sterol RE (SRE)
atcaccccac

SEQ ID NO: 17 - DNA, 13 bp, Artificial Sequence
  Peroxisome proliferator RE (PPRE)
aggtcaaaggtca

SEQ ID NO: 18 - DNA, 9 bp, Artificial Sequence
  Signal transducer and activator of transcription RE (STAT-RE)
ttccaggaa

SEQ ID NO: 19 - DNA, 8 bp, Artificial Sequence
  CACCC-box
ccacaccc

SEQ ID NO: 20 - DNA, 10 bp, Artificial Sequence
  SMAD binding element (SBE)
gtctgcagac

SEQ ID NO: 21 - DNA, 16 bp, Artificial Sequence
  Thyroid response element 1 (TRE1)
aggtcacttcaggtca

SEQ ID NO: 22 - DNA, 18 bp, Artificial Sequence
  Thyroid response element 2 (TRE2)
tgaccttggcataggtca

SEQ ID NO: 23 - DNA, 8 bp, Artificial Sequence
  CCAAT-enhancer binding protein RE (C/EBP-RE)
ttgcgcaa

SEQ ID NO: 24 - DNA, 9 bp, Artificial Sequence
  MCAT
acattcctg

SEQ ID NO: 25 - DNA, 6 bp, Artificial Sequence
  Cellular myeloblastosis RE (cMyb-RE)
taacgg

SEQ ID NO: 26 - DNA, 14 bp, Artificial Sequence
  Heat shock element (HSE)
agaacattctagaa

SEQ ID NO: 27 - DNA, 15 bp, Artificial Sequence
  Estrogen-related receptor RE (ERRE)
aggtcattttgacct

SEQ ID NO: 28 - DNA, 11 bp, Artificial Sequence
  Metal RE (MRE)
tgcacacagcc

SEQ ID NO: 29 - DNA, 10 bp, Artificial Sequence
  D-box
attatgtaac

SEQ ID NO: 30 - DNA, 4 bp, Artificial Sequence
  5′ Linker
ggag

SEQ ID NO: 31 - DNA, 4 bp, Artificial Sequence
  Complementary Linker
cctc

SEQ ID NO: 32 - DNA, 4 bp, Artificial Sequence
  5′ Linker
tact

SEQ ID NO: 33 - DNA, 4 bp, Artificial Sequence
  Complementary Linker
atga

SEQ ID NO: 34 - DNA, 4 bp, Artificial Sequence
  5′ Linker
ccat

SEQ ID NO: 35 - DNA, 4 bp, Artificial Sequence
  Complementary Linker
ggta

SEQ ID NO: 36 - DNA, 4 bp, Artificial Sequence
  5′ Linker
aatg

SEQ ID NO: 37 - DNA, 4 bp, Artificial Sequence
  Complementary Linker
ttac

SEQ ID NO: 38 - DNA, 4 bp, Artificial Sequence
  5′ Linker
aggt

SEQ ID NO: 39 - DNA, 4 bp, Artificial Sequence
  Complementary Linker
tcca

SEQ ID NO: 40 - DNA, 4 bp, Artificial Sequence
  5′ Linker
ttcg

SEQ ID NO: 41 - DNA, 4 bp, Artificial Sequence
  Complementary Linker
aagc

SEQ ID NO: 42 - DNA, 4 bp, Artificial Sequence
  5′ Linker
gctt

SEQ ID NO: 43 - DNA, 4 bp, Artificial Sequence
  Complementary Linker
cgaa

SEQ ID NO: 44 - DNA, 4 bp, Artificial Sequence
  5′ Linker
ggta

SEQ ID NO: 45 - DNA, 4 bp, Artificial Sequence
  Complementary Linker
ccat

SEQ ID NO: 46 - DNA, 4 bp, Artificial Sequence
  5′ Linker
cgct

SEQ ID NO: 47 - DNA, 4 bp, Artificial Sequence
  Complementary Linker
gcga

SEQ ID NO: 48 - DNA, 192 bp, Artificial Sequence
  Low Strength Synthetic Promoter Proximal Region Sequence
tataggaaggtcttaccggaagttccttagctgatagtataccagattttttgcgcaattctagactgatcatctaacgacctattaccggaagttagtatggctagcaggtctatataagcagagctcgtttagtgaaccgtcagatctcaccgtccttgacacgaagcttactgagcacacaggacctgc

SEQ ID NO: 49 - DNA, 215 bp, Artificial Sequence
  Medium Strength Synthetic Promoter Proximal Region Sequence
ttttgcgcaatttataggtggggcggggaaaggtcatgacacagcaatcagatttgcttgcgtgagaagaagtatgttaccggaagttgacctatgggactttccatctaacatgacacagcaatgctagcaggtctatataagcagagctcgtttagtgaaccgtcagatctcaccgtccttgacacgaagcttactgagcacacaggacctgc

SEQ ID NO: 50 - DNA, 291 bp, Artificial Sequence
  High Strength Synthetic Promoter Proximal Region Sequence
tggggcggggaagtatgatgacacagcaattgatcatgggactttccactagactgcttgcgtgagaagaaaggtcttaccggaagttgacctaatgacacagcaatgttagatgcttgcgtgagaagactgatatgggactttccagtatactggggcggggatctaactgggactttccacagattatgacacagcaatgctagcaggtctatataagcagagctcgtttagtgaaccgtcagatctcaccgtccttgacacgaagcttactgagcacacaggacctgc

(19) United States
(12) Patent Application Publication
(10) Pub. No.: US 2024/0294938 A1
(43) Pub. Date: Sep. 5, 2024

(54) CONTROL OF MULTI-GENE EXPRESSION USING SYNTHETIC PROMOTERS

(71) Applicant: ASTRAZENECA AB, Södertälje (SE)

(72) Inventors: Yash Dhananjai PATEL, Sheffield (GB); Suzanne Jane GIBSON, Cambridge (GB); Diane HATTON, Cambridge (GB); Jie ZHU, Gaithersburg, MD (US); David C. JAMES, Sheffield (GB)

(21) Appl. No.: 18/550,548

(22) PCT Filed: Mar. 15, 2022
(86) PCT No.: PCT/IB2022/052323

§ 371 Date: Sep. 14, 2023

Related U.S. Application Data
(60) Provisional application No. 63/200,577, filed on Mar. 16, 2021.

(51) Int. Cl. C12N 15/85

(52) U.S. Cl. CPC C12N 15/85; C12N 2830/48

(57) ABSTRACT

The invention relates to expression vectors comprising mammalian synthetic promoters that can mediate expression of multiple genes at predictable relative stoichiometries.

### CROSS-REFERENCE TO RELATED APPLICATIONS

This application claims benefit under 35 U.S.C. § 119(e) of U.S. Provisional Patent Application No. 63/200,577, filed Mar. 16, 2021, which is incorporated by reference herein in its entirety for all purposes.

### REFERENCE TO SEQUENCE LISTING SUBMITTED ELECTRONICALLY

The content of the electronically submitted sequence listing in ASCII text file (Name: SYNPRO-101-Seq_Listing.txt; Size: 10,840 bytes; and Date of Creation: Mar. 11, 2022) filed with the application is incorporated herein by reference in its entirety.

### BACKGROUND OF THE INVENTION

Mammalian cells utilize complex, finely-tuned gene networks to maintain essential cellular functions. To genetically engineer these networks for biomedical and therapeutic applications, it will ultimately be necessary to precisely control co-expression of multiple recombinant genes simultaneously. While single plasmids encoding multiple transcriptional units (TUs) in series can be constructed using Gibson or Golden Gate assembly technology with relative ease, control of the relative level at which several individual genes are constitutively expressed to achieve a desired stoichiometry is far more difficult. Current methods to achieve controlled expression of recombinant genes in mammalian cells employ multiple single gene synthetic circuits cooperatively functioning using inducible systems and complex gene switches. However, these approaches are problematic in that a limited number of different genes can be controlled, and numerous plasmids have to be co-transfected for stable mammalian cell engineering.

Recombinant gene expression within synthetic circuits can be precisely controlled using an assortment of oscillatory, logic gate and feedback loops. However, this frequently involves the application of synthetic transcription factors, such as transcription activator-like effectors (TALEs), zinc fingers, chimeric transcription factors or CRISPR transcription factors to induce cognate promoters. Alternatively, chemical chaperones, aptamers, metabolites and other external stimuli have all also been employed to induce and regulate synthetic gene circuit expression. These sophisticated biological control systems can be useful, but are also complex and unwieldy, with expression levels determined by ligand (synthetic transcription factors and chemical chaperones) concentration dependent transactivation or repression and the potential of imprecise and leaky expression. Moreover, ligands can inflict a metabolic burden or cellular stress on the host cell, which is undesirable for gene therapy and cell engineering applications. While complex, programmable gene expression systems will be required for many applications, “hardwired” components operating at constitutive fixed stoichiometries generally form the basis of all engineered systems.

An alternative means to control recombinant gene expression stoichiometry is the use of synthetic promoters with defined transcriptional activity. In this case a promoter can be specifically designed to utilize the host cell's existing repertoire of transactivators to a varying extent in order to achieve a desired level of transcriptional activity. As a means to control multi-gene expression stoichiometry, the use of well-defined synthetic promoters in vector constructs is therefore a potentially attractive solution.

### BRIEF SUMMARY OF THE INVENTION

The present invention relates to a multi-gene expression vector comprising a transcription unit comprising a synthetic promoter operably linked to a nucleic acid sequence encoding a nucleotide sequence of interest. In some aspects, the expression vector comprises a second transcription unit comprising a second synthetic promoter operably linked to a nucleic acid sequence encoding a second nucleotide sequence of interest. In some aspects, the expression vector comprises a third transcription unit comprising a third synthetic promoter operably linked to a nucleic acid sequence encoding a third nucleotide sequence of interest.

In some aspects, the first synthetic promoter, second synthetic promoter, and third synthetic promoter have low, medium, or high transcriptional activity. In some aspects, the transcriptional activity of the first synthetic promoter, second synthetic promoter, or third synthetic promoter is repressed by at least 20%, at least 30%, at least 40%, at least 50%, at least 60%, at least 70%, at least 80%, or at least 90% relative to the transcriptional activity from co-expression from a single gene vector. In some aspects, two of the synthetic promoters have the same level of transcriptional activity. In some aspects, the first synthetic promoter, second synthetic promoter, and third synthetic promoter have different levels of transcriptional activity. In some aspects, the first synthetic promoter, second synthetic promoter, and third synthetic promoter have the same level of transcriptional activity. In some aspects, the transcriptional activity is measured by qRT-PCR.

In some aspects, the first synthetic promoter, the second synthetic promoter, and the third synthetic promoter comprises one or more transcription factor regulatory elements (TFREs). In some aspects, the number of TFREs control the transcriptional activity. In some aspects, the low strength synthetic promoter comprises one to three TFREs. In some aspects, the medium strength synthetic promoter comprises four to seven TFREs. In some aspects, the high strength synthetic promoter comprises seven to eleven TFREs. In some aspects, the first synthetic promotor comprises three TFREs, the second synthetic promoter comprises seven TFREs, and the third synthetic promoter comprises eleven TFREs.

In some aspects, the TFREs are selected from the group consisting of ETS binding site (EBS), CCAAT-enhancer binding protein (C/EBP), antioxidant regulatory element (ARE), dioxin regulatory element (DRE), GC-box, and nuclear factor kappa B (NFkB). In some aspects, the low strength synthetic promoter comprises a nucleic acid sequence comprising two EBS and one C/EBP TFREs. In some aspects, the medium strength synthetic promoter comprises a nucleic acid sequence comprising one GC-box, one C/EBP, two ARE, one DRE, one EBS, and one NFkB TFRE. In some aspects, the high strength synthetic promoter comprises a nucleic acid sequence comprising two GC-boxes, three ARE, three NFkB, two DRE, and one EBS TFRE. In some aspects, the expression vector comprises the first transcription unit, the second transcription unit, and the third transcription unit in any orientation.

In some aspects, the first nucleotide sequence of interest, second nucleotide sequence of interest, and third nucleotide sequence of interest are different.

In some aspects, the first transcription unit, second transcription unit, and third transcription unit are joined by nucleic acid linkers. In some aspects, the nucleic acid linkers are selected from the group consisting of SEQ ID NOs: 30-46.

In some aspects, the expression vector is a mammalian, bacterial, or viral expression vector. In some aspects, a cell comprises the expression vector. In some aspects, the cell is a mammalian, bacterial, or plant cell.

Certain aspects of the disclosure are directed to a method of regulating the expression of multiple genes of interest in a cell comprising introducing the expression vector into said cell, and incubating the cell under conditions to promote expression of the nucleotide sequences of interest.

### BRIEF DESCRIPTION OF THE DRAWINGS/FIGURES

FIG. 1A-O show co-expression of three fluorescent reporter proteins to evaluate synthetic promoter activity. FIG. 1A are schematics showing the transcription factor regulatory element (TFRE) composition of the mammalian synthetic promoters. ARE: Antioxidant regulatory element; C/EBP: CCAAT-enhancer binding protein; DRE: Dioxin regulatory element: EBS: ETS binding site; NFkB: Nuclear factor kappa B.

FIG. 1B is a schematic of the pExp-Vec-GG vector comprising of a glutamine synthetase (GS) cassette, mammalian episomal origin of replication, β-lactamase gene for ampicillin resistance and microbial origin of replication that was used as the backbone to assemble an assortment of TUs to construct a MGEV. FIGS. 1C-E are schematics of shuttle plasmids housing the TUs encoding for eGFP under the control of a low (FIG. 1C), medium (FIG. 1D) and high (FIG. 1E) strength synthetic promoter. FIGS. 1F-H are schematics of shuttle plasmids housing the TUs encoding for mCherry under the control of a low (FIG. 1F), medium (FIG. 1G) and high (FIG. 1H) strength synthetic promoter. FIGS. 1I-1K are schematics of shuttle plasmids housing the TUs encoding for tagBFP under the control of a low (FIG. 1I), medium (FIG. 1J) and high (FIG. 1K) strength synthetic promoter. FIG. 1L is a schematic of the low, medium, and high promoters upstream of the three fluorescent reporters (eGFP, mCherry, and tagBFP). FIG. 1M is a graph of synthetic promoter activity determined by relative fluorescent reporter expression fold change. The fold change was derived by normalizing the integrated median fluorescent intensity (iMFI) detected for each reporter utilizing the medium and high strength promoters relative to the low strength promoter. An expression fold change was derived for each total DNA load (100 to 800 ng) transfected and the average fold change for eGFP, mCherry and tagBFP are represented by the white, grey and black bars, respectively. The error bars indicate the standard deviation of reporter expression fold change across all the total DNA loads transfected over three independent experiments. FIG. 1N is a graph of an external calibration curve of the different fluorescent reporter mRNA copies. The calibration curve was derived by arithmetically combining mRNA copies detected at each DNA load (100 to 800 ng) and different promoter strengths while normalizing to the low strength dataset. A third-order polynomial regression curve was fitted to model the different mRNA dynamics of eGFP (Green), mCherry (Red) and tagBFP (Blue) and yield a normalized relative transcriptional activity (RTA) for each promoter-reporter combination. The r² of the third-order polynomial curves for eGFP, mCherry and tagBFP were 0.976, 0.988 and 0.964, respectively. FIG. 1O is a graph of the average fold change in relative transcriptional activity (RTA) of all three fluorescent protein reporters utilizing the medium and high strength synthetic promoters, relative to the low strength promoter mediated expression, at each transfected plasmid load ranging from 100 to 800 ng.

FIG. 2A-C show multi-gene expression vectors (MGEVs) utilizing mammalian synthetic promoters to control recombinant gene expression stoichiometry. FIG. 2A is a schematic showing the library of 27 MGEV variants encoding eGFP, mCherry and tagBFP in a fixed tandem series utilizing a low, medium, and high strength mammalian synthetic promoters in each position encompassing every possible combination. The core promoter and untranslated regions (UTRs) were identical in each transcription unit (TU) within the MGEV-hCMV-MIE core, 5′UTR and SV40 polyA. FIG. 2B is a graph showing the average transcriptional repression of the low, medium, and high strength synthetic promoter exhibited during transient expression of the MGEV library. The percentage of transcriptional repression for each synthetic promoter was calculated by comparing the difference between the RTAs observed during MGEV expression and expected RTAs derived from SGV co-expression at roughly equivalent gene copies. The individual bars and error bars represent the average percentage transcriptional repression and standard deviation respectively for the low, medium, and high strength promoter across all positions within the MGEV (27 discrete RTAs per promoter variant) across three independent experiments. A one-way ANOVA statistical test with a Tukey correction was performed to show a significant difference in average percentage transcriptional repression between the low and medium, and medium and high strength promoters and represented by “****” for p<0.0001. FIG. 2C is a graph showing the average RTA of the low, medium, and high strength synthetic promoter utilized across the 27 discrete MGEV variants irrespective of position. The error bars represent standard deviation of 27 individual RTAs for each promoter across three independent experiments. A one-way ANOVA statistical analysis with a Tukey correction was performed to show significant differences between the low, medium, and high strength synthetic promoters and represented by “****” for p<0.0001.

FIG. 3A-B is an overview of transcriptional activity within a multi-gene expression vector (MGEV) context. FIG. 3A is a three-dimensional plot depicting the relative transcriptional activity (RTA) in position 1, 2 and 3 across the x, y and z axis respectively for 27 discrete MGEV variants utilizing a low, medium, and high strength synthetic promoter in every combination and position within the MGEV and represented as red points. A set of RTAs for a low, medium, and high strength synthetic promoter were derived from single gene vector (SGV) co-expression at approximately equivalent gene copies and compiled to simulate the expected transcriptional activity in each position of a MGEV. These RTAs were represented as blue points on the plot. FIG. 3B is a magnified plot representing the same RTAs in position 1, 2 and 3 of the 27 MGEV variants as shown in (FIG. 3A). The cluster represents the empirically-derived limits of transcriptional activity of the low, medium, and high strength synthetic promoters within the context of a MGEV.

FIG. 4A-B show trends in transcriptional repression within a multi-gene expression vector (MGEV) context. FIG. 4A is a graph of the frequency distribution representing the degree of transcriptional repression across all three positions within the library of 27 MGEV variants. The RTA detected within a MGEV was normalized for overall gene positional effects by compensating for the 15% and 14% repression observed in positions 2 and 3 respectively so to identify other contributing biases in transcriptional activity. These positional effect normalized RTAs for each position and every synthetic promoter combination (low, medium, and high strength) were directly compared against expected RTAs (derived from single gene vector co-expression at approximately equivalent gene copies (FIG. 1L)) to yield a percentage in transcriptional repression. The degree of repression was then categorized into fixed intervals ranging from 0 to 100% as individual bin centers and the frequency calculated for each interval to form a distribution. FIG. 4B is a gradient heat map showing the degree of repression calculated in (FIG. 4A) were arranged according to the synthetic promoter combination and position within the 27 discrete MGEV variants. Shades of purple represent high repression, conversely shades of grey represent lower repression. The synthetic promoter utilized in the specific position is overlaid and abbreviated as “L”, “M” and “H” representing low, medium, and high strength respectively.

### DETAILED DESCRIPTION OF THE INVENTION

Certain aspects of the disclosure are directed to a multi-gene expression vector comprising: (a) a transcription unit comprising a synthetic promoter operably linked to a nucleic acid sequence encoding a nucleotide sequence of interest. In some aspects, the expression vector further comprises a second transcription unit comprising a second synthetic promoter operably linked to a nucleic acid sequence encoding a second nucleotide sequence of interest. In some aspects, the expression vector further comprises a third transcription unit comprising a third synthetic promoter operably linked to a nucleic acid sequence encoding a third nucleotide sequence of interest.

In some aspects, the first synthetic promoter, second synthetic promoter, and third synthetic promoter individually have low, medium, or high transcriptional activity. In some aspects, the three promoters cause the same transcriptional activity. In some aspects, the transcriptional activity of the three promoters is different. In some aspects, the transcriptional activity is measured by qRT-PCR.

In some aspects, the first synthetic promoter, the second synthetic promoter, and the third synthetic promoter comprises one or more transcription factor regulatory elements (TFREs). In some aspects, the number of TFREs control the transcriptional activity. In some aspects, the low strength synthetic promoter comprises one to three TFREs. In some aspects, wherein the medium strength synthetic promoter comprises four to seven TFREs. In some aspects, the high strength synthetic promoter comprises seven to eleven TFREs. In some aspects, the first synthetic promotor comprises three TFREs, the second synthetic promoter comprises seven TFREs, and the third synthetic promoter comprises eleven TFREs.

In some aspects, the TFREs can be ETS binding site (EBS), CCAAT-enhancer binding protein (C/EBP), antioxidant regulatory element (ARE), dioxin regulatory element (DRE), GC-box, and nuclear factor kappa B (NFkB). In some aspects, the low strength synthetic promoter comprises a nucleic acid sequence comprising two EBS and one C/EBP TFREs. In some aspects, the medium strength synthetic promoter comprises a nucleic acid sequence comprising one GC-box, one C/EBP, two ARE, one DRE, one EBS, and one NFkB TFRE. In some aspects, the high strength synthetic promoter comprises a nucleic acid sequence comprising two GC-boxes, three ARE, three NFkB, two DRE, and one EBS TFRE.

In some aspects, the expression vector comprises the first transcription unit, the second transcription unit, and the third transcription unit in any orientation. In some aspects, the first nucleotide sequence of interest, second nucleotide sequence of interest, and/or third nucleotide sequence of interest are different. In some aspects, the first nucleotide sequence of interest, second nucleotide sequence of interest, and/or third nucleotide sequence of interest are the same.

In some aspects, the expression vector is a mammalian, bacterial, or viral expression vector. In some aspects, a cell comprises the expression vector. In some aspects, the cell is a mammalian, bacterial, or plant cell.

Certain aspects of the disclosure are directed to a method of regulating the expression of multiple genes of interest in a cell comprising introducing the expression vector into said cell, and incubating the cell under conditions to promote expression of the nucleotide sequences of interest.

### I. Definitions

In order that the present disclosure can be more readily understood, certain terms are first defined. Additional definitions are set forth throughout the detailed disclosure.

The term “promoter” as used herein defines a regulatory DNA sequence that mediates the initiation of transcription by directing RNA polymerase to bind to DNA and initiating RNA synthesis. Promoters are generally located upstream of a gene. A promoter can comprise, for example, a core promoter and transcription factor regulatory elements.

A “synthetic promoter” refers to an artificial, engineered, and/or assembled promoter comprising transcription factor regulatory elements.

A “transcription factor regulatory element” (TFRE) is a nucleotide sequence that is a binding site for a transcription factor. Exemplary TFREs are provided in Table 1.

A “transcription factor” (TF) is a protein that binds to a TFRE and affects the rate of transcription (either positively or negatively) of a gene.

A “core promoter” refers to a nucleotide sequence that is the minimal portion of the promoter required to initiate transcription. Core promoter sequences can be derived from prokaryotic or eukaryotic genes, including, e.g., the CMV immediate early gene promoter or SV40. A core promoter can comprise, for example, a TATA box. A core promoter can comprise, for example, an initiator element. A core promoter can comprise, for example, a TATA box and an initiator element.

The term “enhancer” as used herein defines a nucleotide sequence that acts to potentiate the transcription of a gene, independent of the identity of the gene, the position of the sequence in relation to the gene, and the orientation of the sequence.

The terms “functionally linked” and “operably linked” are used interchangeably and refer to a functional relationship between two or more DNA segments (e.g., a gene to be expressed and a sequence(s) controlling the gene's expression). For example, a promoter and/or enhancer sequence, including any combination of cis-acting transcriptional control elements, is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Orientation” refers to the order of nucleotides in a given DNA sequence. For example, an orientation of a DNA sequence in opposite direction in relation to another DNA sequence is one in which the 5 ‘ to 3’ order of the sequence in relation to another sequence is reversed when compared to a point of reference in the DNA from which the sequence was obtained. Such reference points can include the direction of transcription of other specified DNA sequences in the source DNA and/or the origin of replication of replicable vectors containing the sequence.

The term “expression vector” as used herein includes an isolated and purified DNA molecule which upon transfection into an appropriate host cell provides for expression of a recombinant gene product within the host cell. In addition to the DNA sequence coding for the recombinant or gene product the expression vector comprises regulatory DNA sequences that are required for an efficient transcription of the DNA coding sequence into mRNA and optionally for an efficient translation of the mRNAs into proteins in the host cell line.

The terms “host cell” or “host cell line” as used herein include any cells, in particular mammalian cells, which are capable of growing in culture and expressing a desired recombinant product protein.

The term “expression cassette” as used herein includes a polynucleotide sequence encoding a polypeptide to be expressed and sequences controlling its expression such as a promoter and optionally an enhancer sequence, including any combination of cis-acting transcriptional control elements. The sequences controlling the expression of the gene, i.e. its transcription and the translation of the transcription product, are commonly referred to as regulatory unit. Most parts of the regulatory unit are located upstream of coding sequence of the gene and are operably linked thereto. The expression cassette may also contain a downstream 3′-untranslated region comprising a polyadenylation site. The regulatory unit of the invention is either operably linked to the gene to be expressed, i.e. transcription unit, or is separated therefrom by intervening DNA such as for example by the 5′-untranslated region of the heterologous gene. The expression cassette can be flanked by one or more suitable restriction sites in order to enable the insertion of the expression cassette into a vector and/or its excision from a vector. The expression cassette can comprises one or more suitable restriction sites in order to enable the insertion or deletion of different genetic elements (i.e., TFREs, nucleic acid sequences of interest, promoters, terminators, etc.). Thus, the expression cassette according to the present invention can be used for the construction of an expression vector, in particular a mammalian expression vector. The expression cassette of the present invention may comprise one or more e.g. two, three or even more non-translated genomic DNA sequences downstream of a promoter.

The terms “polynucleotide” and “nucleotide sequence” include naturally occurring nucleic acid molecules or recombinantly expressed nucleic acid molecules, which can be isolated from a cell, as well as synthetic molecules, which can be prepared, for example, by methods of chemical synthesis or by enzymatic methods such as polymerase chain reaction (PCR).

The term “polynucleotide sequence encoding a polypeptide” as used herein includes DNA coding for a gene, preferably a heterologous gene expressing the polypeptide.

The terms “heterologous coding sequence”, “heterologous gene sequence”, “heterologous gene”, “recombinant gene” or “gene” are used interchangeably. These terms refer to a DNA sequence that codes for a recombinant, in particular a recombinant heterologous protein product that is sought to be expressed in a host cell, preferably in a mammalian cell and harvested. The product of the gene can be a polypeptide. The heterologous gene sequence is naturally not present in the host cell and is derived from an organism of the same or a different species and may be genetically modified.

The terms “protein” and “polypeptide” are used interchangeably to include a series of amino acid residues connected to the other by peptide bonds between the alpha-amino and carboxy groups of adjacent residues.

The term “high strength synthetic promoter” as used herein refers to a promoter which is able to express a recombinant gene, recombinant protein, or reporter protein at a “high” level of expression, defined as a level of expression that is higher than the mean level of expression obtained across a range of promoter constructs.

The term “medium strength synthetic promoter” as used herein refers to a promoter which is able to express a recombinant gene, recombinant protein, or reporter protein at a “medium” level of expression, defined as a level of expression that is equal than the mean level of expression obtained across a range of promoter constructs.

The term “low strength synthetic promoter” as used herein refers to a promoter which is able to express a recombinant gene, recombinant protein, or reporter protein at a “low” level of expression, defined as a level of expression that is lower than the mean level of expression obtained across a range of promoter constructs.

The term “nucleotide sequence of interest” as used herein can be any nucleic acid that encodes a protein or other molecule that is desirable for expression in a host cell (e.g., for production of the protein or other biological molecule (e.g., a therapeutic cellular product) in the target cell).

The term “stoichiometry” as used herein refers to the relative quantities of proteins expressed by an expression vector.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein the term “linker” refers to a chemical moiety that connects a molecule to another molecule. The linker provides spacing between the two molecules or moieties such that they are able to function in their intended manner.

As used herein the term “transcription unit” refers to a nucleic acid sequence comprising a regulatory unit (i.e., a synthetic promoter) operably linked to a nucleotide sequence of interest (i.e., a gene to be expressed).

### II. Expression Vectors

Using the methods provided herein, mammalian expression vectors can be designed containing synthetic promoters that mediate expression of multiple nucleotide sequences of interest at predictable relative stoichiometries. Exemplary synthetic promoters are described in International Patent Application No. PCT/EP2018/060125, which is herein incorporated by reference in its entirety. The nucleotide sequences of interest can be any nucleotide sequence that encodes a protein or other molecule that is desirable for expression in a host cell. In some aspects, the expression vector comprises a transcription unit comprising a nucleic acid sequence encoding a nucleic acid sequence of interest. In some aspects, the expression vector can comprise a first transcription unit comprising a first nucleic acid sequence encoding a first nucleic acid sequence of interest and a second transcription unit comprising a second nucleic acid sequence encoding a second nucleic acid sequence of interest. In some aspects, the expression vector can comprise a first transcription unit comprising a first nucleic acid sequence encoding a first nucleic acid sequence of interest, a second transcription unit comprising a second nucleic acid sequence encoding a second nucleic acid sequence of interest, and a third transcription unit comprising a third nucleic acid sequence encoding a third nucleic acid sequence of interest. In some aspects, the expression vector comprises more than three transcription units. In some aspects, the expression vector comprises at least three transcription units, at least four transcription units, at least five transcription units, at least six transcription units, at least seven transcription units, or at least eight transcription units. In some aspects, the first nucleotide sequence of interest, second nucleotide sequence of interest, and/or third nucleotide sequence of interest are different. In some aspects, the first nucleotide sequence of interest, second nucleotide sequence of interest, and/or third nucleotide sequence of interest are the same. In some aspects, the expression vector comprises the first transcription unit, the second transcription unit, and the third transcription unit in any orientation.

In some aspects, the expression vector comprises one or more synthetic promoters. In some aspects, the transcription unit comprises one or more synthetic promoters. In some aspects, the expression vector comprises three synthetic promoters. In some aspects, the first synthetic promoter, second synthetic promoter, and third synthetic promoter have low, medium, or high transcriptional activity. In some aspects, the transcriptional activity of the first synthetic promoter, second synthetic promoter, and third synthetic promoter is repressed by at least 20%, at least 30%, at least 40%, at least 50%, at least 60%, at least 70%, at least 80%, or at least 90% relative to the transcriptional activity from co-expression from a single gene vector. In some aspects, two of the synthetic promoters have the same level of transcriptional activity. In some aspects, the first synthetic promoter, second synthetic promoter, and third synthetic promoter have different levels of transcriptional activity. In some aspects, the first synthetic promoter, second synthetic promoter, and third synthetic promoter have the same level of transcriptional activity. In some aspects, the transcriptional activity is measured by qRT-PCR or RNA sequencing. In some aspects, the transcriptional activity is measured by qRT-PCR.

In some aspects, the expression vector comprises a transcription unit comprising a synthetic promoter operably linked to a nucleic acid sequence encoding a nucleic acid sequence of interest. In some aspects, the expression vector can comprise a first transcription unit comprising a first synthetic promoter operably linked to a first nucleic acid comprising a first nucleic acid sequence encoding a first nucleic acid sequence of interest and a second transcription unit comprising a second synthetic promoter operably linked to a second nucleic acid sequence encoding a second nucleic acid sequence of interest. In some aspects, the expression vector can comprise a first transcription unit comprising a first synthetic promoter operably linked to a first nucleic acid sequence encoding a first nucleic acid sequence of interest, a second transcription unit comprising a second synthetic promoter operably linked to a second nucleic acid sequence encoding a second nucleic acid sequence of interest, and a third transcription unit comprising a third synthetic promoter operably linked to a third nucleic acid sequence encoding a third nucleic acid sequence of interest. In some aspects, the first synthetic promoter, the second synthetic promoter, and the third synthetic promoter comprises one or more transcription factor regulatory elements (TFREs). In some aspects, the number of TFREs control the transcriptional activity.

A synthetic promoter can comprise any number and any combination of TFREs. In some aspects, the low strength synthetic promoter comprises one to three TFREs. In some aspects, the low strength promoter comprises three TFREs. In some aspects, the medium strength synthetic promoter comprises four to seven TFREs. In some aspects, the medium strength synthetic promoter comprises seven TFREs. In some aspects, the high strength synthetic promoter comprises seven to eleven TFREs. In some aspects, the high strength synthetic promoter comprises eleven TFREs. In some aspects, wherein the first synthetic promotor comprises three TFREs, the second synthetic promoter comprises seven TFREs, and the third synthetic promoter comprises eleven TFREs.

Exemplary transcription factor regulatory elements, include but are not limited to, ETS binding site, CCAAT-enhancer binding protein, antioxidant regulatory element (ARE), dioxin regulatory element (DRE), nuclear factor kappa B (NFkB), GC-box and those listed in Table 1.

TABLE 1
Transcription factor regulatory element consensus sequences.

| Transcription Factor Response/ | |
|---|---|
| Regulatory Element (RE) | Sequence (SEQ ID NO.) |
| Enhancer box (E-box) | CACGTG (SEQ ID NO: 1) |
| cAMP RE (CRE) | TGACGTCA (SEQ ID NO: 2) |
| Amino acid RE (AARE) | ATTGCATCA (SEQ ID NO: 3) |
| Forkhead box binding site (FBS) | ATAAACAA (SEQ ID NO: 4) |
| Nuclear factor kappa B (NFkB-RE) | GGGACTTTCC (SEQ ID NO: 5) |
| Nuclear factor 1 RE (NF1-RE) | TTGGCTATATGCCAA (SEQ ID NO: 6) |
| Antioxidant RE (ARE) | ATGACACAGCAAT (SEQ ID NO: 7) |
| Elongation factor 2 RE (E2F-RE) | TTTCGCGC (SEQ ID NO: 8) |
| GC-box | GGGGCGGGG (SEQ ID NO: 9) |
| ETS binding site 1 (EBS1) | ACCGGAAGT (SEQ ID NO: 10) |
| ETS binding site 2 (EBS2) | ACAGGAAGT (SEQ ID NO: 11) |
| Unfolded protein response element (UPRE) | GCTGACGTGGTGCTGACGTGG (SEQ |
| | ID NO: 12) |
| Endoplasmic reticulum stress RE II (ERSE-II) | ATTGGTCCACG (SEQ ID NO: 13) |
| Yin Yang 1 RE (YY1-RE) | CGCCATTTT (SEQ ID NO: 14) |
| Endoplasmic reticulum stress RE (ERSE) | CCAATGGCCAGCCTCCACG (SEQ ID |
| | NO: 15) |
| Sterol RE (SRE) | ATCACCCCAC (SEQ ID NO: 16) |
| Peroxisome proliferator RE (PPRE) | AGGTCAAAGGTCA (SEQ ID NO: 17) |
| Signal transducer and activator of | TTCCAGGAA (SEQ ID NO: 18) |
| transcription RE (STAT-RE) | |
| CACCC-box | CCACACCC (SEQ ID NO: 19) |
| SMAD binding element (SBE) | GTCTGCAGAC (SEQ ID NO: 20) |
| Thyroid response element 1 (TRE1) | AGGTCACTTCAGGTCA (SEQ ID |
| | NO: 21) |
| Thyroid response element 2 (TRE2) | TGACCTTGGCATAGGTCA (SEQ ID |
| | NO: 22) |
| CCAAT-enhancer binding protein RE | TTGCGCAA (SEQ ID NO: 23) |
| (C/EBP-RE) | |
| MCAT | ACATTCCTG (SEQ ID NO: 24) |
| Cellular mycloblastosis RE (cMyb-RE) | TAACGG (SEQ ID NO: 25) |
| Heat shock element (HSE) | AGAACATTCTAGAA (SEQ ID NO: 26) |
| Estrogen-related receptor RE (ERRE) | AGGTCATTTTGACCT (SEQ ID NO: 27) |
| Metal RE (MRE) | TGCACACAGCC (SEQ ID NO: 28) |
| D-box | ATTATGTAAC (SEQ ID NO: 29) |

In some aspects, the transcription units are joined by nucleic acid linkers. In some aspects, the first transcription unit is joined to the second transcription unit by a nucleic acid linker. In some aspects, the second transcription unit is joined to the third transcription unit by a nucleic acid linker. In some aspects, the nucleic acid linker is between 1 and 10 base pairs in length, between 2 and 8 base pairs in length, or between 3 and 6 base pairs in length. In some aspects, the nucleic acid linker is 4 base pairs in length. Nucleic acid linkers include, but are not limited to, the linkers listed in Table 2.

TABLE 2
Nucleic Acid Linker Sequences

| | Complementary Linker |
|---|---|
| 5′ Linker (SEQ ID NO) | (SEQ ID NO) |
| 5′-GGAG-3′ (SEQ ID NO: 30) | 3′-CCTC-5′ (SEQ ID NO: 31) |
| 5′-TACT-3′ (SEQ ID NO: 32) | 3′-ATGA-5′ (SEQ ID NO: 33) |
| 5′-CCAT-3′ (SEQ ID NO: 34) | 3′-GGTA-5′ (SEQ ID NO: 35) |
| 5′-AATG-3′ (SEQ ID NO: 36) | 3′-TTAC-5′ (SEQ ID NO: 37) |
| 5′-AGGT-3′ (SEQ ID NO: 38) | 3′-TCCA-5′ (SEQ ID NO: 39) |
| 5′-TTCG-3′ (SEQ ID NO: 40) | 3′-AAGC-5′ (SEQ ID NO: 41) |
| 5′-GCTT-3′ (SEQ ID NO: 42) | 3′-CGAA-5′ (SEQ ID NO: 43) |
| 5′-GGTA-3′ (SEQ ID NO: 44) | 5′-CCAT-5′ (SEQ ID NO: 45) |
| 5′-CGCT-3′ (SEQ ID NO: 46) | 3′-GCGA-5′ (SEQ ID NO: 47) |

In some aspects, low strength synthetic promoter comprises a nucleic acid sequence comprising two EBS and one C/EBP TFREs. In some aspects, the low strength promoter comprises a nucleic acid sequence having at least 50% identity, at least 60% identity, at least 70% identity, at least 75% identity, at least 80% identity, at least 85% identity, at least 90% identity, at least 95% identity, or 100% identity to SEQ ID NO: 30. In some aspects, the medium strength synthetic promoter comprises a nucleic acid sequence comprising one C/EBP, one GC-box, two ARE, one DRE, one EBS, and one NFkB TFRE. In some aspects, the low strength promoter comprises a nucleic acid sequence having at least 50% identity, at least 60% identity, at least 70% identity, at least 75% identity, at least 80% identity, at least 85% identity, at least 90% identity, at least 95% identity, or 100% identity to SEQ ID NO: 31. In some aspects, the high strength synthetic promoter comprises a nucleic acid sequence comprising three ARE, three NFkB, two DRE, two GC-box and one EBS TFRE. In some aspects, the low strength promoter comprises a nucleic acid sequence having at least 50% identity, at least 60% identity, at least 70% identity, at least 75% identity, at least 80% identity, at least 85% identity, at least 90% identity, at least 95% identity, or 100% identity to SEQ ID NO: 32.

TABLE 3
Low, Medium, and High Strength Synthetic Promoter Proximal Region Sequences

| Description | |
|---|---|
| (SEQ ID NO) | Sequence |
| Low Strength Synthetic | TATAGGAAGGTCTTACCGGAAGTTCCTTAGCTGATA |
| Promoter Proximal | GTATACCAGATTTTTTGCGCAATTCTAGACTGATCAT |
| Region Sequence (SEQ ID | CTAACGACCTATTACCGGAAGTTAGTATGGCTAGCA |
| NO: 48) | GGTCTATATAAGCAGAGCTCGTTTAGTGAACCGTCA |
| | GATCTCACCGTCCTTGACACGAAGCTTACTGAGCAC |
| | ACAGGACCTGC |
| Medium Strength | TTTTGCGCAATTTATAGGTGGGGGGGGGAAAGGTCA |
| Synthetic Promoter | TGACACAGCAATCAGATTTGCTTGCGTGAGAAGAAG |
| Proximal Region | TATGTTACCGGAAGTTGACCTATGGGACTTTCCATCT |
| Sequence (SEQ ID NO: | AACATGACACAGCAATGCTAGCAGGTCTATATAAGC |
| 49) | AGAGCTCGTTTAGTGAACCGTCAGATCTCACCGTCC |
| | TTGACACGAAGCTTACTGAGCACACAGGACCTGC |
| High Strength Synthetic | TGGGGGGGGGAAGTATGATGACACAGCAATTGATC |
| Promoter Proximal | ATGGGACTTTCCACTAGACTGCTTGCGTGAGAAGAA |
| Region Sequence (SEQ ID | AGGTCTTACCGGAAGTTGACCTAATGACACAGCAAT |
| NO: 50) | GTTAGATGCTTGCGTGAGAAGACTGATATGGGACTT |
| | TCCAGTATACTGGGGGGGGGATCTAACTGGGACTTT |
| | CCACAGATTATGACACAGCAATGCTAGCAGGTCTAT |
| | ATAAGCAGAGCTCGTTTAGTGAACCGTCAGATCTCA |
| | CCGTCCTTGACACGAAGCTTACTGAGCACACAGGAC |
| | CTGC |

The vector of the invention encompasses viral as well as non-viral (e.g. plasmid DNA) vectors. Suitable non-viral vectors include, but are not limited to, plasmids such as pExp-Vec-GG, pExp-Vec-GG (GS only), pExp-Vec-GG (OriP only), pExp-Vec-GG (TI), pExp-Vec-GG (Hygro), and pExp-Vec-GG (GS+UCOE). A “viral vector” is used herein according to its art-recognized meaning. It refers to any vector that comprises at least one element of viral origin, including a complete viral genome, a portion thereof or a modified viral genome as described below as well as viral particles generated thereof (e.g. viral vector packaged into a viral capsid to produce infectious viral particles). Viral vectors of the invention can be replication-competent, or can be genetically disabled so as to be replication-defective or replication-impaired. In some aspects, the expression vector is a mammalian, bacterial, or viral expression vector.

A cell can comprise a promoter provided herein or a vector provided herein. The cell can be a mammalian cell, such as a Chinese Hamster Ovary (CHO) cell. The CHO cell can be, for example, a CHO-S, CHO-K1, CHO-DG44, or a CHO-DXB11 cell. The cell can be, for example, a human cell. The cell can also be, for example, a non-human cell, e.g., a non-human mammalian cell. A cell comprising a promoter or vector provided herein can be transiently transfected or stably transfected. A cell provided herein can be an isolated cell (i.e. a cell not contained within an organism) or a cultured cell (i.e. a cell in culture).

### Examples

### Example 1: Transient Co-Expression of Reporter Genes Using Synthetic Promoters in a Single Gene Per Plasmid Vector Format

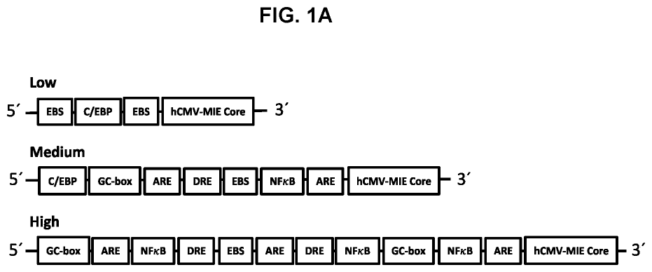

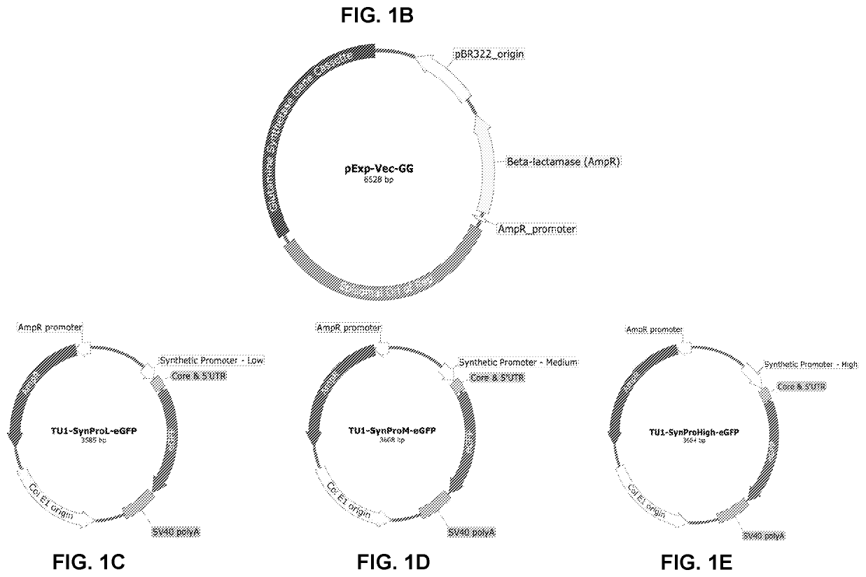

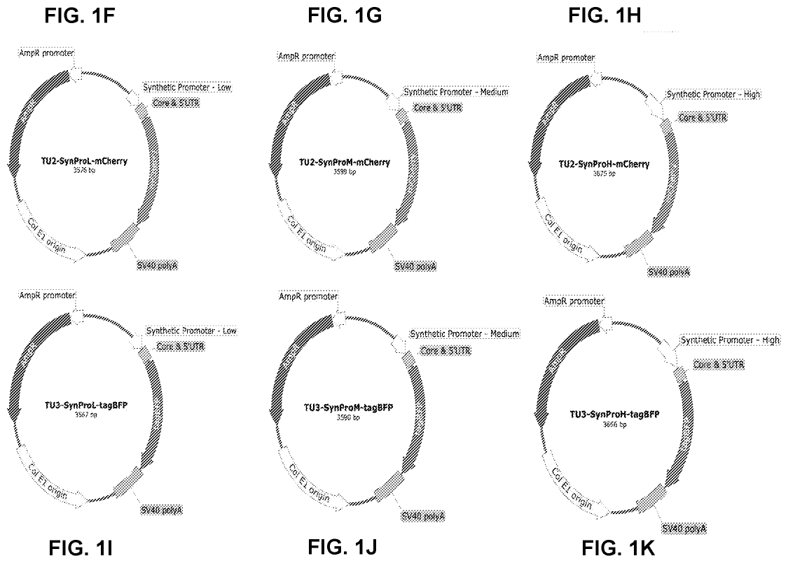

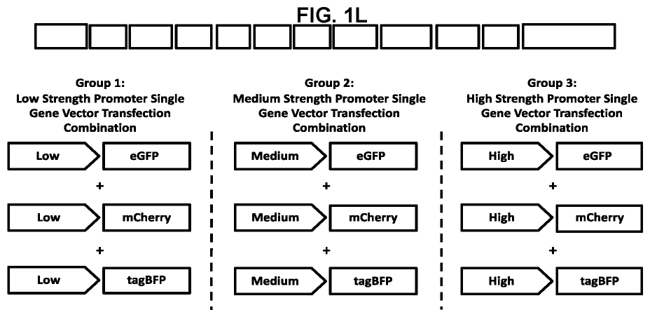

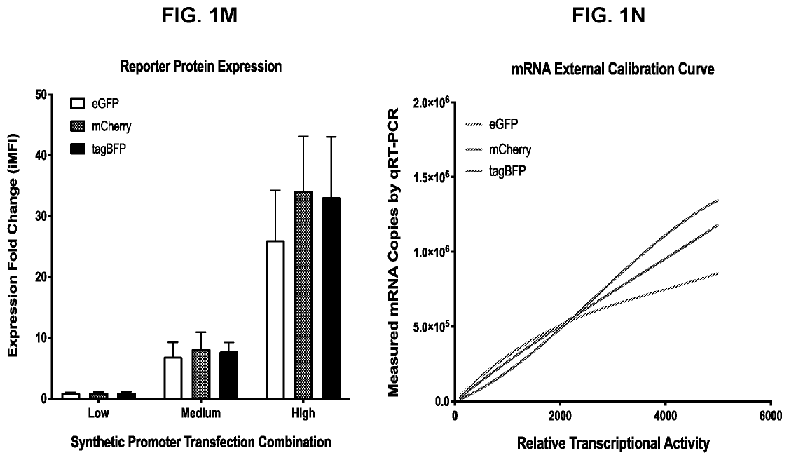

Synthetic promoter transcription factor regulatory element (TFRE) composition is shown in FIG. 1A. Promoters were comprised of a selection of up to seven different TFREs varying in transcriptional activity as previously characterized in CHO cells. The TFRE blocks separated by a 2 bp spacer were specifically selected for the low, medium, and high strength promoters and positioned upstream of the human cytomegalovirus major intermediate-early (hCMV-MIE) core in order to vary each promoter's transcriptional activity. The low, medium, and high strength synthetic promoter's approximate activity was 0.1, 0.8 and 2.2-fold of hCMV-MIE expression strength respectively.

In order to confirm synthetic promoter transcriptional activity and quantitatively evaluate recombinant gene expression from single gene vectors (SGVs) for subsequent comparison with gene expression from MGEVs, each synthetic promoter was inserted upstream of three spectrally discrete fluorescent reporter proteins, eGFP, mCherry and tagBFP to create a library of nine SGVs (FIGS. 1C-1L). These were co-transfected in three groups, each group consisting of three SGVs encoding each fluorescent protein under the control of the same transcriptionally active promoter, either low (group 1), medium (group 2), or high (group 3), at a total plasmid DNA load ranging from 100 to 800 ng (FIG. 1L). SGV-mediated transient expression of each reporter gene resulted in a relatively low, medium, or high cellular content of fluorescent protein dependent upon the synthetic promoter utilized. Across all reporters, normalized (relative to low strength promoter data) integrated median fluorescence intensities were in the ratio 1:7.7:31.2 (low:medium:high strength synthetic promoters), confirming expected promoter functionality (FIG. 1M).

However, as the sensitivity of flow cytometric detection of fluorescent proteins at low expression levels was limited, and in order to quantitatively compare transcriptional activity more directly, recombinant cellular reporter mRNA content was measured using absolute quantitation qRT-PCR. In order to externally calibrate measured mRNA copies to variation in transcriptional activity for each reporter, recombinant mRNA copies derived from a range of transfected SGV total plasmid DNA loads (over the range 100 to 800 ng per 1.86×10⁶ cells) were measured by qRT-PCR. For each experiment an equal mass of eGFP, mCherry and tagBFP SGVs utilizing either low, medium, or high strength promoters were mixed prior to transfection such that all reporters were co-transfected as either low, medium, or high strength synthetic promoter groups as previously described (FIG. 1L). As each reporter gene was a similar length (eGFP, 720 bp; mCherry, 711 bp; tagBFP, 702 bp) the number of copies of each reporter gene co-transfected in each experiment was similar, (e.g. 600 ng total plasmid DNA equals 26983-27868 copies of each fluorescent reporter gene per cell). At each total SGV DNA load, reporter mRNA copies were measured 24 h after transfection by qRT-PCR. To enable direct comparison of transcriptional activities for each reporter mRNA measurement, mRNA copies measured at each DNA load and varying promoter strength were arithmetically combined and normalized with respect to the low strength promoter datasets, incorporating the assumption that whilst reporter-specific mRNA dynamics likely vary post-transcription (i.e. mRNA half-life, mRNA secondary structure, translation efficiency etc.) the transcriptional rate mediated by a given promoter was constant for each reporter gene. This enabled reporter-specific external calibration curves to be created, relating measured mRNA copies to a cross-reporter comparable relative transcriptional activity (RTA). In each case, a third-order polynomial regression provided the line of best fit (r² of 0.976, 0.988 and 0.964 for eGFP, mCherry and tagBFP calibrations respectively; FIG. 1N). Unsurprisingly, reporter-specific differences in measured mRNA copies and RTAs were apparent, indicative of differences in mRNA dynamics despite the use of common 5′ and 3′ UTRs in each case. Across all SGV data (accounting for every total DNA load), synthetic promoters yielded RTAs in the normalized mean (±SE) ratio low 1:medium 4.6 (±1.1):high 7.2 (±1.1). The ratio of synthetic promoter activities was to an extent dependent upon total plasmid DNA load, such that relative to the low strength promoter, the medium, and high ratios increased linearly with mass of transfected DNA (FIG. 1O), potentially indicative of reduced self-inhibition (also referred to as promoter interference) with increased promoter complexity at higher DNA loads.

### Example 2: Construction and Performance of Multi-Gene Expression Vectors Utilizing Synthetic Promoters to Control Recombinant Gene Expression Stoichiometry

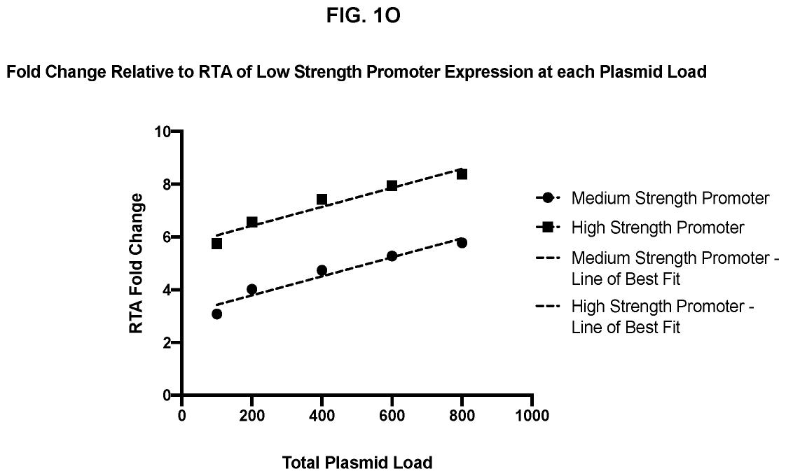

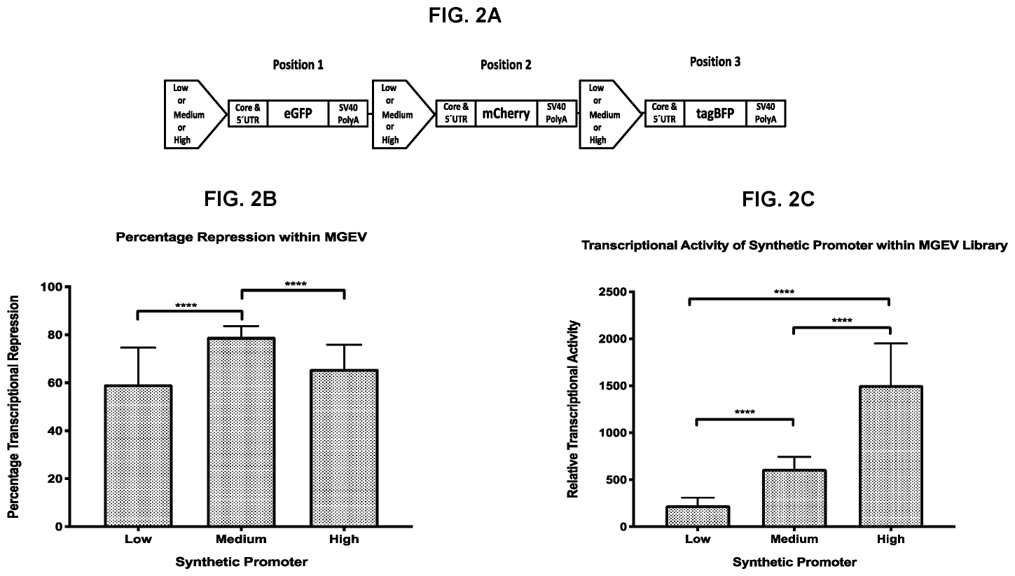

Synthetic promoters were tested to see if they could be used to predictably control the relative level of expression of recombinant genes arranged in series in MGEVs. A library of 27 MGEVs encoding eGFP, mCherry and tagBFP in a fixed series utilizing all possible combinations of synthetic promoters (low, medium, and high) in the different positions was constructed (FIG. 2A). Each MGEV was constructed by Golden Gate assembly using the de novo synthesized TUs and plasmid vector backbone pExp-Vec-GG (FIGS. 1B-1K).

Each MGEV variant was transfected into CHO cells for 24 h as per the SGV combinations (FIG. 1L) at a total MGEV mass of 600 ng per 1.86×10⁶ cells. Under these conditions, the number of fluorescent gene copies transfected (29113±212 copies of each fluorescent reporter gene per cell) was approximately equivalent to the number of gene copies transfected using 600 ng of combined SGV vector plasmid DNA (27489 copies of each reporter per cell, see above). Therefore, derived from SGV expression data at the same plasmid DNA load employed, the predicted RTAs for low, medium, and high strength synthetic promoters respectively were 552 (±20), 2915 (±284) and 4384 (±874) respectively, a ratio of 1:5.3:7.9. For each MGEV, reporter mRNA copies were measured by qRT-PCR and RTAs derived using the SGV external calibration (FIG. 1N). These data are listed in Table 4. The RTA was derived by interpolating against the single gene vector (SGV) external calibration curves (FIG. 1N) to allow direct comparison of reporter expression. The low, medium, and high strength synthetic promoters utilized in each position of the MGEV variants was abbreviated to “L”, “M,” and “H”, respectively. A mean RTA for position 1, 2 and 3 across the 27 MGEV variants was calculated and an overall gene positional effect ratio was derived by normalizing the mean RTA in position 2 and 3 relative to position 1.

TABLE 4
Average Relative Transcriptional Activity of the Reporter mRNAs
Synthetic Promoter Variant/Average
Relative Transcriptional Activity (±Standard Deviation)

| Position 1 | | | Position 2 | | | Position 3 | | |
|---|---|---|---|---|---|---|---|---|
| L | 206.0 | (±10.1) | L | 244.0 | (±8.5) | L | 114.1 | (±8.0) |
| L | 172.7 | (±19.2) | L | 224.6 | (±29.9) | M | 685.8 | (±138.5) |
| L | 211.6 | (±13.2) | L | 311.1 | (±20.9) | H | 2158.1 | (±353.6) |
| L | 149.4 | (±20.3) | M | 450.3 | (±77.7) | L | 96.2 | (±18.5) |
| L | 177.1 | (±24.4) | M | 748.6 | (±108.8) | M | 479.6 | (±65.2) |
| L | 229.3 | (±19.4) | M | 877.5 | (±49.3) | H | 1762.7 | (±53.2) |
| L | 224.8 | (±40.5) | H | 1331.0 | (±179.9) | L | 249.1 | (±36.4) |
| L | 272.4 | (±32.8) | H | 1616.2 | (±148.0) | M | 469.6 | (±32.1) |
| L | 219.1 | (±31.6) | H | 1444.7 | (±315.3) | H | 949.7 | (±191.4) |
| M | 557.9 | (±76.2) | L | 265.8 | (±31.0) | L | 100.1 | (±16.6) |
| M | 491.0 | (±99.8) | L | 251.1 | (±33.8) | M | 469.1 | (±91.0) |
| M | 583.6 | (±44.9) | L | 323.2 | (±14.2) | H | 1883.8 | (±308.5) |
| M | 647.2 | (±71.5) | M | 477.1 | (±53.9) | L | 139.5 | (±18.8) |
| M | 904.8 | (±149.1) | M | 698.8 | (±86.4) | M | 679.5 | (±83.6) |
| M | 713.2 | (±111.4) | M | 741.0 | (±102.8) | H | 2001.6 | (±143.5) |
| M | 596.6 | (±18.7) | H | 974.3 | (±63.3) | L | 185.7 | (±5.1) |
| M | 845.5 | (±65.3) | H | 1469.1 | (±95.1) | M | 549.0 | (±55.4) |
| M | 593.0 | (±20.0) | H | 1211.1 | (±50.6) | H | 1028.2 | (±81.2) |
| H | 2058.5 | (±234.2) | L | 414.4 | (±11.0) | L | 169.5 | (±2.6) |
| H | 1333.4 | (±130.7) | L | 399.8 | (±23.1) | M | 502.6 | (±29.5) |
| H | 1189.8 | (±144.3) | L | 375.6 | (±14.0) | H | 1189.1 | (±69.9) |
| H | 1686.2 | (±202.2) | M | 547.3 | (±32.6) | L | 172.9 | (±20.3) |
| H | 1862.9 | (±173.6) | M | 614.7 | (±23.5) | M | 469.4 | (±22.9) |
| H | 1117.9 | (±270.9) | M | 790.5 | (±27.1) | H | 1487.1 | (±74.8) |
| H | 1201.3 | (±50.5) | H | 674.6 | (±17.5) | L | 164.6 | (±6.8) |
| H | 1950.6 | (±243.9) | H | 1062.2 | (±32.2) | M | 473.4 | (±7.9) |
| H | 2655.2 | (±355.3) | H | 1411.5 | (±120.5) | H | 1736.6 | (±141.9) |

Average Relative Transcriptional Activity for each position

| | | |
|---|---|---|
| 862.9 | 734.0 | 744.5 |

Average Positional Effect Ratio

| | | |
|---|---|---|
| 1.00 | 0.85 | 0.86 |

The general trend observed was a substantial overall repression of reporter gene transcription relative to that observed using SGVs (overall mean of 69.9% relative to SGV mediated transcription). This repression is quantified per synthetic promoter in FIG. 2B and a comparison of relative promoter-mediated transcriptional activity is shown in FIG. 2C (in both cases across all combinations utilized). These data show that in a MGEV context synthetic promoters did generally yield the expected transcriptional trend (i.e. L<M<H; FIG. 2C), although the actual ratio (1:2.8:6.7) was different from that obtained using SGVs (1:5.3:7.9). Together, the data show more marked repression of the medium strength synthetic promoter (FIG. 2B). It can be inferred that overall transcriptional repression may be attributed to a change in plasmid structure by negative supercoiling, where the plasmid conformation post-RNA polymerase II (RNA pol II) transcription elongation can hinder upstream gene transcription. Additionally, the potential bidirectional behavior of promoters in a fixed tandem series can lead to antisense transcription and RNA pol II collisions, in turn inhibiting transcription of neighboring TUs. Both these mechanisms could be concurrently contributing towards the general repression within a MGEV context.

Gene positional effect within the library of vectors was quantified simply by summation of all RTAs deriving from positions 1, 2, and 3 (i.e. using all synthetic promoter combinations; Table 4). This revealed that maximum reporter expression occurred at position 1. Relative to position 1, positions 2 and 3 exhibited a 15% and 14% reduction in reporter gene transcription respectively. Therefore, the gene positional effect may be a consequence of inefficient transcription termination of the upstream TU causing transcriptional read through of the RNA pol II elongation complex into the neighboring TU. This limits binding of transcription factors or assembly of the pre-initiation complex by steric hindrance in the promoter region of the TU inhibiting transcription initiation. The mechanism is referred to as occlusion-mediated transcriptional interference. Alternatively, the RNA pol II elongation complex can also dislodge transcription factors bound to the enhancer region of a downstream promoter resulting in repressed transcription. Other dual promoter systems in tandem arrangement within a standard or lentiviral vector have also exhibited unpredictable gene expression both caused by transcriptional interference. Similarly, a triple gene cassette constructing a synthetic pathway in Saccharomyces cerevisiae also exhibited substantial discrepancies from predicted expression attributed to transcriptional interference.

### Example 3: Bias in Recombinant Gene Transcription in the Multigene Vector Context

The observed RTA for each of the 27 MGEV variants (Table 4) was further analyzed to discern specific biases in recombinant gene transcription within a multi-gene context. This was achieved by comparing the “observed RTA” within a MGEV against a set of “expected RTAs” derived from SGV co-expression at approximately equivalent gene copies.

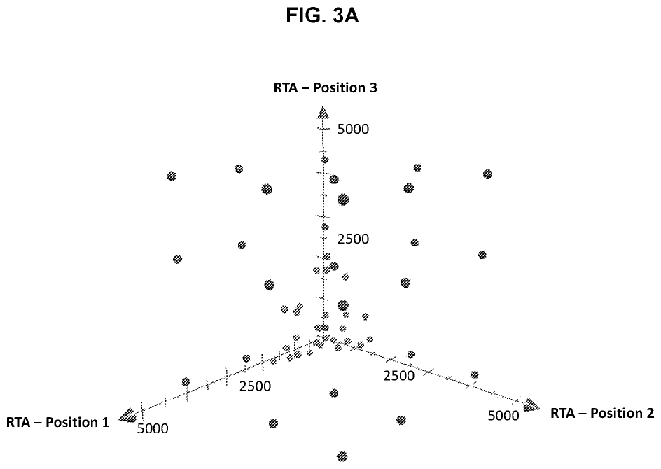

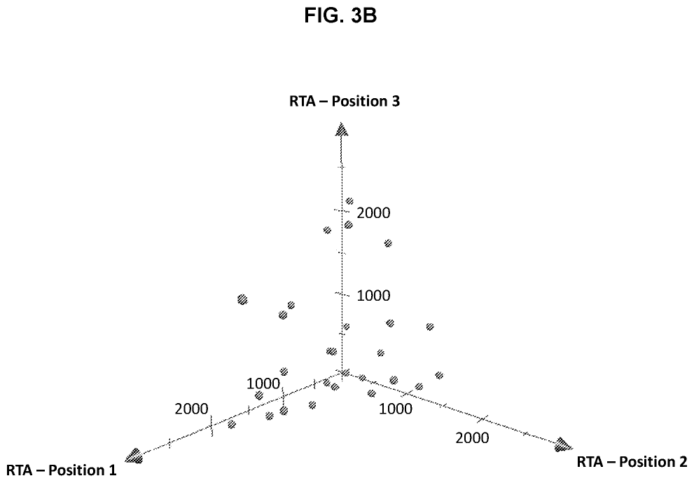

The observed RTA of each position within a MGEV was compared to its expected RTA counterpart within a three-dimensional plot as shown in FIG. 3, where each axis represented one of the three positions within the MGEV. FIG. 3A reiterates the substantial transcriptional repression in all positions for each MGEV as shown by the clustered conformation of the observed RTAs (ranging from 96.2 to 2655.2) compared to the cubic conformation of the expected RTA (ranging from 551.9 to 4383.5). The cluster of MGEV RTAs (FIG. 3B) was asymmetrical with lower overall transcriptional activity observed in position 2 across 27 discrete variants (mean RTA of 734.0) re-emphasizing increased general transcriptional repression compared to position 1 and 3. FIG. 3B depicts the empirically-derived design space for achievable transcriptional activity of three recombinant genes in a fixed tandem series utilizing a low, medium, and high strength synthetic promoter accounting for a range of potential transcriptional interfering mechanisms.

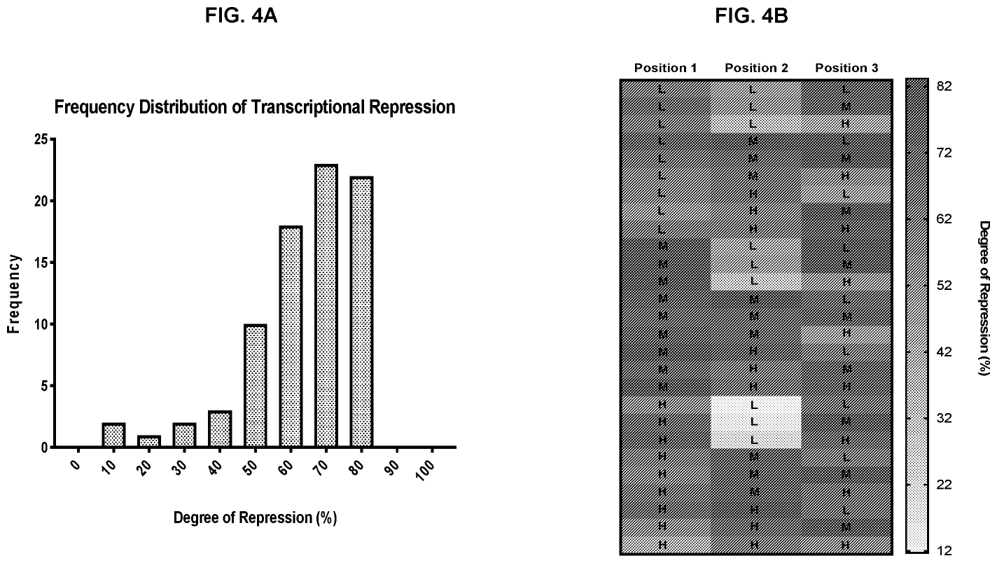

In order to identify other transcriptional repression trends within the MGEV library, the overall observed gene positional effect (15% and 14% repression in position 2 and 3 respectively) were normalized for the detected RTA in position 2 and 3 of each MGEVs. These positional effect normalized RTAs from MGEV expression were compared against expected RTAs (derived from SGV co-expression at roughly equivalent gene copies) yielding a percentage of transcriptional repression for each position. The distribution of transcriptional repression irrespective of position showed majority (90.1%) of expression was repressed by >50% and the median transcriptional repression was 68.6% (FIG. 4A).

Positional or promoter specific gene repression trends were highlighted in FIG. 4B. The color gradient heat map depicts the degree of repression relative to the expected RTAs for each position across the MGEV library. The medium strength synthetic promoter consistently demonstrated repressed activity with an average transcriptional repression of 76.5% (12% higher than the mean transcriptional repression observed). Conversely, the low strength synthetic promoter exhibited enhanced transcriptional activity when neighboring a higher strength synthetic promoter where the degree of repression (48.2%) was lower than the average (64.5%). The promoter activity was particularly higher in position 2 with average transcription repression of 31.7%. Generally, the high strength promoter did not exhibit any specific transcriptional trends but broad context-specific variation was evident where repression ranged from 39.4 to 81.9%.

The deviation of promoter activity (after accounting for general repression) is context-specific to the localized environment within a MGEV where promoter squelching may be impacting transcription. Promoter squelching refers to competition of transcription factors and associated cofactors involved in regulating transcription between promoter variants resulting in bias gene expression activity. When referring to the TFRE composition of the low, medium, and high strength synthetic promoter, all six transcription factors and their cognate TFREs are shared between the promoter variants (FIG. 1A). The medium strength synthetic promoter shares TFRE-blocks with both the low (EBS and C/EBP) and high (GC-box, ARE, DRE, EBS, NFkB) strength synthetic promoter indicating increased competition of transcription factors. This would suggest the repressed state of the medium strength promoter is potentially caused by squelching. The enhanced activity of low strength synthetic promoter neighboring a higher strength synthetic promoter variant could be caused by interaction between transcription factors.

What is claimed is:

1. A multi-gene expression vector comprising a transcription unit comprising a synthetic promoter operably linked to a nucleic acid sequence encoding a nucleotide sequence of interest.

2. The multi-gene expression vector of claim 1, further comprising a second transcription unit comprising a second synthetic promoter operably linked to a nucleic acid sequence encoding a second nucleotide sequence of interest.

3. The multi-gene expression vector of claim 1 or claim 2, further comprising a third transcription unit comprising a third synthetic promoter operably linked to a nucleic acid sequence encoding a third nucleotide sequence of interest.

4. The multi-gene expression vector of claims 1-3, wherein the first synthetic promoter, second synthetic promoter, and third synthetic promoter have low, medium, or high transcriptional activity.

5. The multi-gene expression vector of claim 4, wherein the transcriptional activity of the first synthetic promoter, second synthetic promoter, or third synthetic promoter is repressed by at least 20%, at least 30%, at least 40%, at least 50%, at least 60%, at least 70%, at least 80%, or at least 90% relative to the transcriptional activity from co-expression from a single gene vector.

6. The multi-gene expression vector of claim 4, wherein two of the synthetic promoters have the same level of transcriptional activity.

7. The multi-gene expression vector of claim 4, wherein the first synthetic promoter, second synthetic promoter, and third synthetic promoter have different levels of transcriptional activity.

8. The multi-gene expression vector of claim 4, wherein the first synthetic promoter, second synthetic promoter, and third synthetic promoter have the same level of transcriptional activity.

9. The multi-gene expression vector of claim 4, wherein the transcriptional activity is measured by qRT-PCR.

10. The multi-gene expression vector of claims 1-3, wherein the first synthetic promoter, the second synthetic promoter, and the third synthetic promoter comprises one or more transcription factor regulatory elements (TFREs).

11. The multi-gene expression vector of claim 9, wherein the number of TFREs control the transcriptional activity.

12. The multi-gene expression vector of claim 10, wherein the low strength synthetic promoter comprises one to three TFREs.

13. The multi-gene expression vector of claim 10, wherein the medium strength synthetic promoter comprises four to seven TFREs.

14. The multi-gene expression vector of claim 10, wherein the high strength synthetic promoter comprises seven to eleven TFREs.

15. The multi-gene expression vector of claim 10, wherein the first synthetic promotor comprises three TFREs, the second synthetic promoter comprises seven TFREs, and the third synthetic promoter comprises eleven TFREs.

16. The multi-gene expression vector of claim 9, wherein the TFREs are selected from the group consisting of ETS binding site (EBS), CCAAT-enhancer binding protein (C/EBP), antioxidant regulatory element (ARE), dioxin regulatory element (DRE), GC-box, and nuclear factor kappa B (NFkB).

17. The multi-gene expression vector of claim 15, wherein the low strength synthetic promoter comprises a nucleic acid sequence comprising two EBS and one C/EBP TFREs.

18. The multi-gene expression vector of claim 15, wherein the medium strength synthetic promoter comprises a nucleic acid sequence comprising one GC-box, one C/EBP, two ARE, one DRE, one EBS, and one NFkB TFRE.

19. The multi-gene expression vector of claim 15, wherein the high strength synthetic promoter comprises a nucleic acid sequence comprising two GC-boxes, three ARE, three NFkB, two DRE, and one EBS TFRE.

20. The multi-gene expression vector of claims 1-3, wherein the expression vector comprises the first transcription unit, the second transcription unit, and the third transcription unit in any orientation.

21. The multi-gene expression vector of claims 1-3, wherein the first nucleotide sequence of interest, second nucleotide sequence of interest, and third nucleotide sequence of interest are different.

22. The multi-gene expression vector of claims 1-3, wherein the first transcription unit, second transcription unit, and third transcription unit are joined by nucleic acid linkers.

23. The multi-gene expression vector of claim 21, wherein the nucleic acid linkers are selected from the group consisting of SEQ ID NOs: 30-46.

24. The multi-gene expression vector of claim 1, wherein the expression vector is a mammalian, bacterial, or viral expression vector.

25. A cell comprising the multi-gene expression vector of any of the preceding claims.

26. The cell of claim 24, wherein the cell is a mammalian, bacterial, or plant cell.

27. A method of regulating the expression of multiple genes of interest in a cell comprising introducing the expression vector of claim 1 into said cell, and incubating the cell under conditions to promote expression of the nucleotide sequences of interest.
